(19) United States
(12) Patent Application Publication
(10) Pub. No.: US 2008/0319348 A1
(43) Pub. Date: Dec. 25, 2008

(54) DIAGNOSIS CATHETER FOR INTERSTITIAL CYSTITIS

(75) Inventors: Yasuhiko Nakajima, Kanagawa (JP); Tomohiro Ueda, Kyoto (JP)

Correspondence Address: HARNESS, DICKEY & PIERCE, P.L.C., P.O. BOX 828, BLOOMFIELD HILLS, MI, 48303, US

(73) Assignee: Tsukada Medical Research Co., Ltd., Tokyo (JP)

(21) Appl. No.: 12/133,957

(22) Filed: Jun. 5, 2008

(30) Foreign Application Priority Data
Jun. 21, 2007 (JP) 163554/2007

(51) Int. Cl. A61B 5/20; A61B 18/18; A61M 25/00

(52) U.S. Cl. 600/585; 606/41

(57) ABSTRACT

Provided is a diagnosis catheter 101 for an interstitial cystitis wherein a catheter 101 is connected to a current perception threshold inspection apparatus 4 to diagnose the interstitial cystitis, comprising: a cylindrical catheter body 111 made of a soft flexible material and including a distal end section A to be left in the bladder 102 and a body section B disposed adjacent to the distal end section A and defined in a proximal end side; at least one electrode 114 provided on a distal end of the catheter body 111; and a lead wire 115 inserted into the catheter body 111 so that first end of the lead wire is connected to the electrode 114 and second end of the lead wire is drawn out of a proximal end of the catheter body 111 to be connected to the current perception threshold inspection apparatus 4.

### BACKGROUND OF THE INVENTION

1. Field of the Invention

This invention relates to a diagnosis catheter for an interstitial cystitis and more particularly relates to a catheter with electrodes to be used for diagnosis of an interstitial cystitis.

2. Prior Art

A catheter with electrodes is well known and is generally used for diagnosis or treatment of a neurotic cystitis, an acraturesis, or the like. However, there is no example in which the catheter with the electrodes has been used for diagnosis of the interstitial cystitis. Since a conventional catheter with electrodes has exposed hard electrodes, it is not suitable for insertion into a bladder through a urethra. There is another type of catheter with an inflatable balloon mounted on an outer periphery around a catheter body for the purpose of fixedly holding the catheter in place.

There is a current perception threshold (CPT) inspection apparatus that has been recently developed to diagnose an abnormality of a peripheral nerve. This inspection apparatus evaluates an amount of a current stimulation at the lowest level that a subject can feel when a feeble alternating current is supplied to a pair of electrodes stuck on a skin of the subject. The CPT inspection apparatus is generally utilized in a measurement of a treatment effect (anesthesia), a measurement of an affected portion (plastic surgery), an evaluation of a diabetic peripheral neuropathy (medicine), a quantitative evaluation of a perceptive nerve (neurology), a diagnosis for distinguishing a disease between an impotence due to a neuropathy and a psychogenetic impotence (urology), an evaluation of an injury and a perception (dentistry), a quantitative measurement of a pharmacodynamics effect (pharmacology), and the like.

The above CPT inspection apparatus is also used in a urology to diagnose a urinary organ outside a body of the subject. However, the CPT inspection apparatus has not been used for diagnosis of the interstitial cystitis. The interstitial cystitis is a disease that has been found recently. A deterministic diagnosis has not been found yet, although there are many latent patients.

The interstitial cystitis is often caused in women in the ages of 20 to 60. The interstitial cystitis often appears as a symptom such as a pain in an upper part of a pubic bone, a thamuria, an urge uresiesthesia, or the like. In a typical mucosa observation, a canker-appears in line-like shapes on a bladder mucosa. Even in a lighter symptom, a spot bleeding appears on a substantially wide area of the bladder mucosa. A general inflammation is a phenomenon that occurs between the time when a tissue is injured and the time when the injured tissue heals. However, the tissue continues to heal in the interstitial cystitis.

Since pathology of the interstitial cystitis has not been resolved yet, a common reference of diagnosis has not been proposed. There are various common methods for diagnosing the interstitial cystitis, such as an endoscopy using a cystoscope, an observation of an interior in the bladder utilizing an inflation by means of a water pressure, a bladder biopsy in which an inflammatory tissue is removed and inspected outside a body of a subject, and the like. However, any one of the above diagnosis methods is not convenient and precise.

In the light of the current circumstances as described above, the applicant of the present invention had submitted the patent application entitled “Diagnosis Catheter for interstitial cystitis” directed to a balloon catheter with an electrode, which had been internationally laid-open under International Publication No. WO2004/043260 on May 27, 2004 and later registered under the Patent No. 3921221 or Feb. 23, 2007. The diagnosis catheter for interstitial cystitis comprises two electrodes mounted on an outer periphery of a catheter body, each of which electrodes is connected with a current perception threshold inspection apparatus via a lead wire. In an actual practice for a clinical diagnosis, predetermined levels of current is applied between the electrodes, and a value for the current level perceptible by a patient is recorded, from which the diagnosis is given whether the patient has interstitial cystitis or not (see cited Patent document 1).

### SUMMARY OF THE INVENTION

An initial symptom of the interstitial cystitis is a hypersensitivity in a urethra and/or a bladder. There is a method for injecting a KCl (potassium chloride) into a bladder in one of conventional simple inspection methods. However, this method induces a pain in a subject and the pain continues after injection of the KCl. This method is not suitable for a low injury inspection method. The KCl is a serious matter in the low injury inspection for the interstitial cystitis that can be moderated by filling a C-fiber into the bladder. To this end, the applicant of the present invention has developed the diagnosis catheter for interstitial cystitis as disclosed in the above cited Patent document 1.

However, the catheter as illustrated in FIG. 6 of the Patent document 1 is usable for giving a diagnosis in the cervical part leading to the urethra from the bladder but not for the diagnosis on the interior wall of the bladder.

In addition, to give an giagnosis across a large area of the bladder interior wall, a distal end section of the catheter body is required to be bent previously. Unfortunately, this may introduce another drawback that the catheter that is previously bent is difficult to insert and advance into the bladder.

An object of the present invention is to provide a diagnosis catheter for an interstitial cystitis that can diagnose the interstitial cystitis on the interior wall of the bladder simply and precisely and does not cause a pain in a subject.

Another object of the present invention is to provide a diagnosis catheter for an interstitial cystitis that is also usable for giving a diagnosis across a large area of the bladder interior wall.

To accomplish the above objects, an invention as defined in claim 1 has employed a configuration for a diagnosis catheter for an interstitial cystitis wherein the catheter is connected to a current perception threshold inspection apparatus to diagnose an interstitial cystitis, in which the catheter comprises: a cylindrical catheter body made of a soft flexible material and including a distal end section to be left in the bladder and a body section disposed adjacent to the distal end section and defined in a proximal end side; a pair of electrodes provided on a distal end of the catheter body; and lead wires inserted into the catheter body so that first ends of the lead wires are connected to the electrodes and second ends of the lead wires are drawn out of a proximal end of the catheter body to be connected to the current perception threshold inspection apparatus.

Further, a diagnosis catheter for an interstitial cystitis according to an invention as defined in claim 2 has employed a configuration, in which the catheter comprises: a balloon mounted on an outer periphery of the catheter body near a boundary between the distal end section and the body section; a fluid supply passage formed inside the catheter body so that an end of the passage is in communication with the balloon; and an injection section capable of injecting a predetermined fluid through the fluid supply passage.

Yet further, a diagnosis catheter for an interstitial cystitis according to an invention as defined in claim 3 has employed a configuration, in which both end sections of the balloon adhere to an exterior surface of the catheter body, and a width of adhesion varies circumferentially along the exterior surface of the catheter body.

Still further, a diagnosis catheter for an interstitial cystitis according to an invention as defined in claim 4 has employed a configuration, in which the balloon has a thickness varying circumferentially along the exterior surface of the catheter body.

Yet further, a diagnosis catheter for an interstitial cystitis according to an invention as defined in claim 5 has employed a configuration, in which the catheter body has an exterior wall thickness varying circumferentially along the exterior surface of the catheter body.

A diagnosis catheter for an interstitial cystitis according to the present invention, owing to an arrangement with the electrode being provided on the distal end of the catheter body, makes it possible to diagnose the interior wall of the bladder, as well. In addition, the catheter body can be bent by injecting a predetermined volume of fluid (e.g., between 2 cc to 5 cc) into the balloon and an angle of bending can be set desirably (e.g. between 0 to 40 degrees) in dependence on the volume of fluid injection, so that the catheter can help give a diagnosis in a site across a large area of the bladder interior wall.

### BRIEF DESCRIPTION OF THE DRAWINGS

FIG. 1 is a schematic longitudinal sectional view of a diagnosis catheter for an interstitial cystitis according to the prior art that is disclosed by way of reference example.

FIG. 2 is a cross sectional view of the catheter taken along line II-II in FIG. 1.

FIG. 3 is a cross sectional view of the catheter taken along line III-III in FIG. 1.

FIG. 4 is a cross sectional view of the catheter taken along line IV-IV in FIG. 1.

FIGS. 5(A) to 5(C) are partial plan views of a portion of the catheter as disclosed in FIG. 1, illustrating various modifications of electrodes of the catheter.

FIG. 6 is an explanatory view of an example in which the catheter as disclosed in FIG. 1 is used for diagnosis of an interstitial cystitis.

FIGS. 7(A) and 7(B) are graphs illustrating a diagnosis result of each interstitial cystitis indicated on a display of a current perception threshold inspection apparatus, FIG. 7(A) illustrating a normal value, and FIG. 7(B) illustrating an abnormal value due to the interstitial cystitis, respectively.

FIGS. 8(A) and 8(B) show a diagnosis catheter for an interstitial cystitis according to a first embodiment of the present invention, FIG. 8(A) illustrating a front view, and FIG. 8(B) illustrating a longitudinal cross sectional view, respectively.

FIG. 9(A) is a sectional view illustrating a diagnostic procedure being carried out in the bladder with the diagnosis catheter for an interstitial cystitis as disclosed in FIG. 8 and FIG. 9(B) is a sectional view illustrating a catheter having a modified electrode.

FIGS. 10(A) and 10(B) are sectional views of a diagnosis catheter for an interstitial cystitis according to a second embodiment of the present invention, FIG. 10(A) illustrating a condition with a balloon not in an expanded configuration and FIG. 10(B) illustrating another condition with the balloon in an expanded configuration and biasing the catheter body to bend.

FIG. 11 is a sectional view illustrating the diagnostic procedure being carried out in the bladder with the diagnosis catheter for an interstitial cystitis as disclosed in FIG. 10.

FIGS. 12(A) and 12(B) are sectional views illustrating a diagnosis catheter for an interstitial cystitis according to a variation of the second embodiment, FIG. 12(A) illustrating a longitudinal sectional view and FIG. 12(B) illustrating a sectional view taken along B-B line of FIG. 12(A).

FIGS. 13(A) and 13(B) are sectional views of a diagnosis catheter for an interstitial cystitis according to another variation of the second embodiment of the present invention, FIG. 13(A) illustrating a longitudinal sectional view and FIG. 13(B) illustrating a sectional view taken along B-B line of FIG. 13(A).

FIG. 14 is a front view illustrating a diagnosis catheter for an application in vagina.

### DETAILED DESCRIPTION OF THE PREFERRED EMBODIMENTS

### REFERENCE EXAMPLE

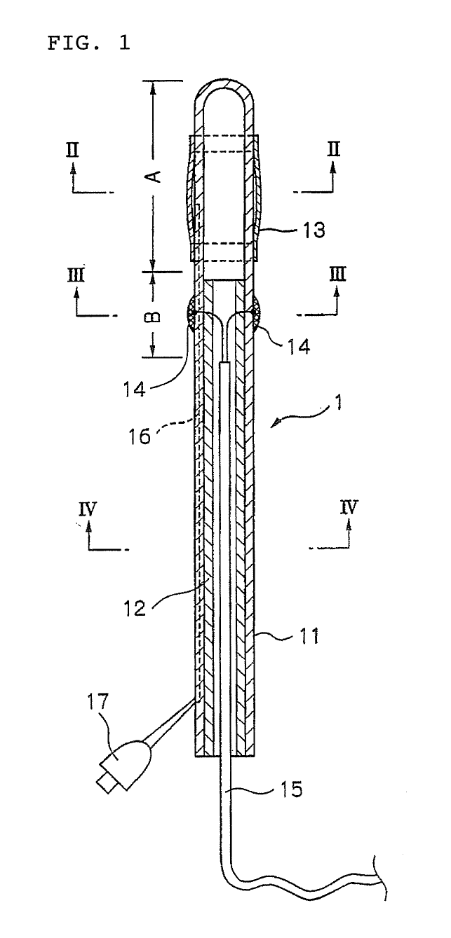

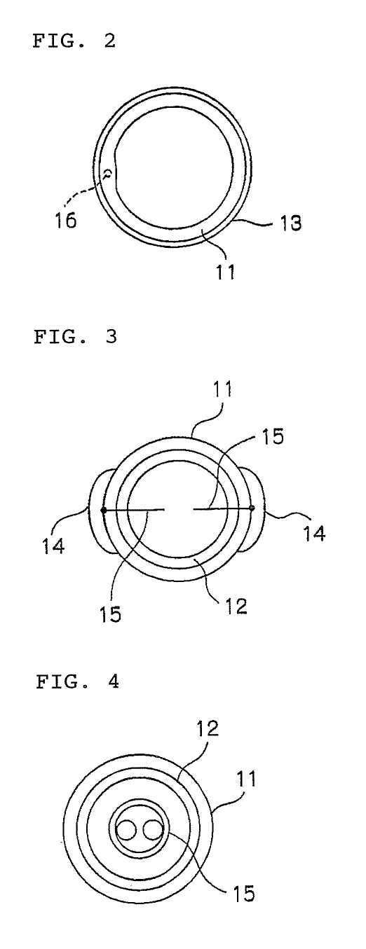

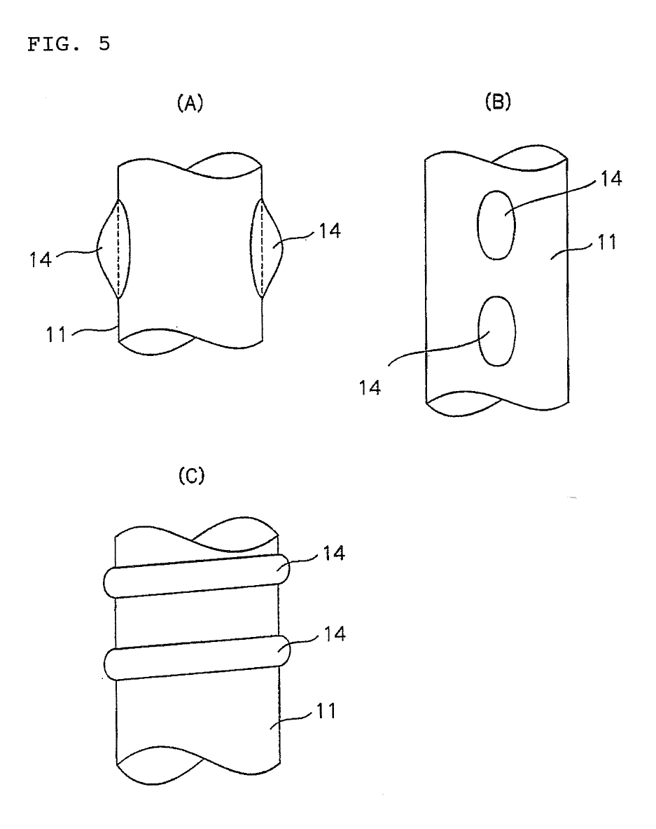

Since a diagnosis catheter for an interstitial cystitis according to the present invention is an improvement of a catheter as disclosed in the Patent document 1 by the common applicant, firstly the catheter of the Patent document 1 will be described with reference to FIGS. 1 to 7, as reference examples.

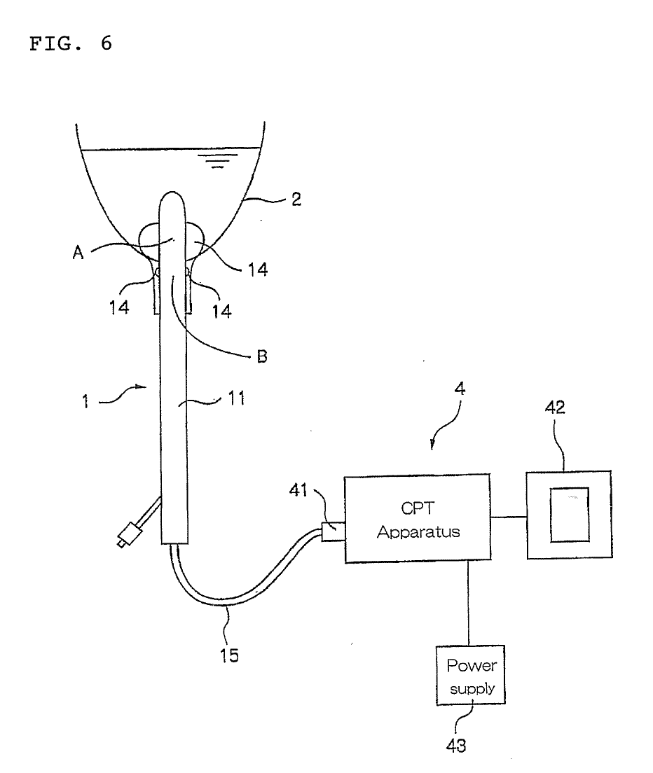

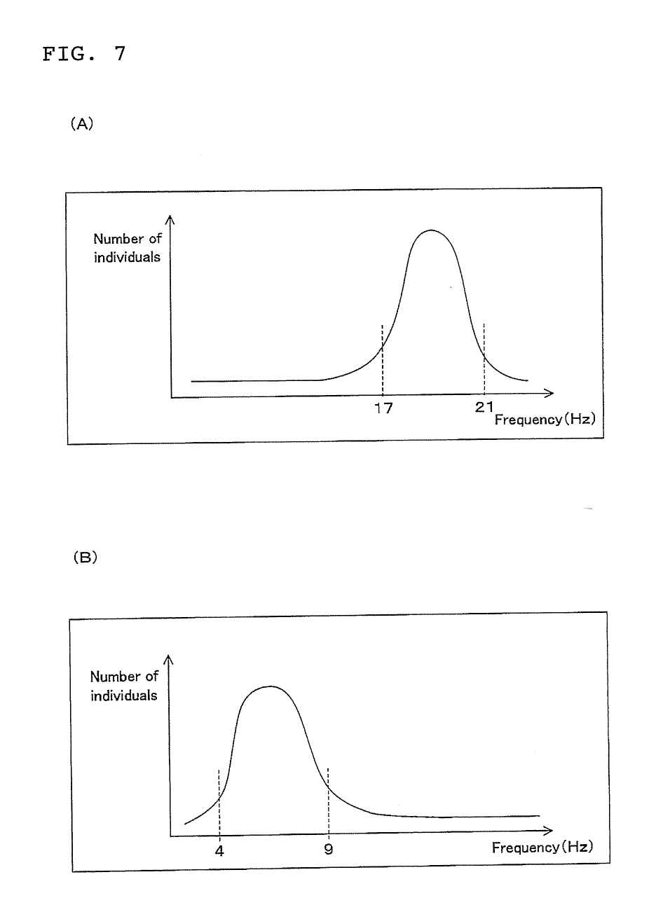

Referring first to FIGS. 1 to 5, an embodiment of a diagnosis catheter 1 for an interstitial cystitis in accordance with the present invention will be described below. As shown in FIG. 1, the diagnosis catheter 1 for the interstitial cystitis in accordance with the present invention is connected to a current perception threshold inspection apparatus 4 (CPT inspection apparatus) shown in FIG. 6 and is used to diagnose the interstitial cystitis.

The diagnosis catheter 1 for the interstitial cystitis in accordance with the present invention includes mainly a catheter body 11, a core member 12, an inflatable balloon 13, a pair of electrodes 14, lead wires 15, and a fluid supply passage 16.

The catheter body 11 is made of a soft flexible material (for example, silicone rubber) and includes a bladder-dwelling distal end section A and a diagnosis section B adjacent to the distal end section A at a proximal end side of the body 11. The core member 12 is made of a hard flexible material (for example, polytetrafluoroethylene resin) and is inserted into the diagnosis section B in the catheter body 11. The inflatable balloon 13 is mounted on an outer periphery around the bladder-dwelling distal end section A of the catheter body 11.

The pair of electrodes 14 is provided on an outer periphery of the diagnosis section B. The lead wires 15 are inserted into the core member 12 so that first ends of the lead wires 15 are connected to the electrodes 14 and second ends of the lead wires 15 are drawn out of a proximal end of the catheter body 11 to be connected to the current perception threshold inspection apparatus 4.

The fluid supply passage 16 is provided in the catheter body 11 so that an end of the passage 16 is communicated to the balloon 13 and a proximal end of the passage 16 is communicated to an injection part 17. A fluid (for example, air, water, or the like) is supplied from (through) the injection part 17 (through the passage 16) to the balloon 13 to inflate the balloon 13.

The electrodes 14, as shown in FIG. 5, may be separated away from each other in a diametrical direction of the catheter body (FIG. 5(A)) or in an axial direction of the catheter body (FIG. 5(B)). Otherwise, the electrodes 14 may be ring-like electrodes that extend continuously in a peripheral direction of the catheter body 11 and separated away form each other in an axial direction of the body 11 (FIG. 5(C)).

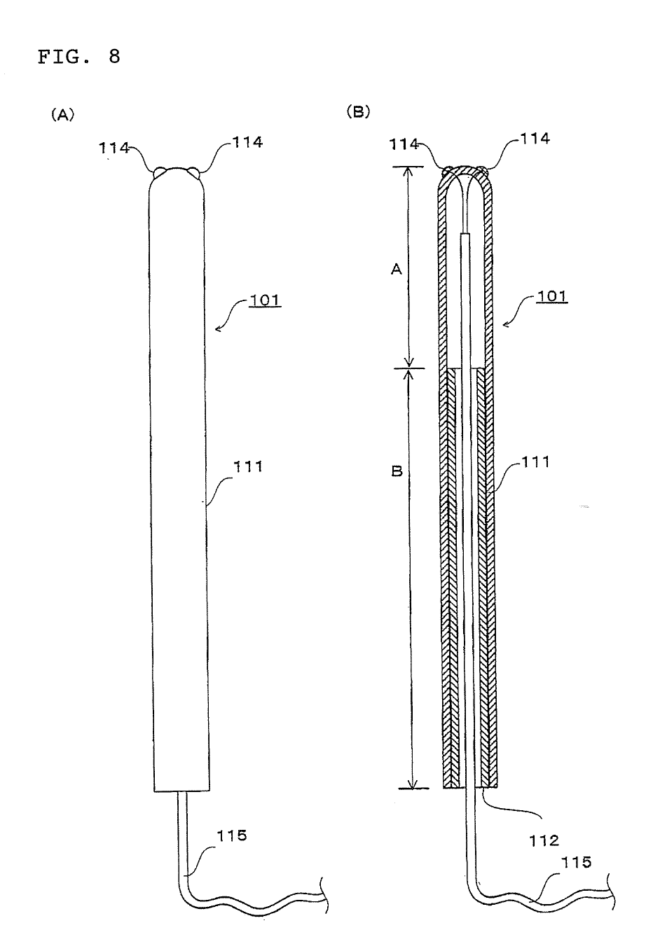

Referring secondly to FIGS. 6 and 7, an example of diagnosis of the interstitial cystitis utilizing the catheter 1 of the present invention will be explained below.

As shown in FIG. 6, the catheter 1 is inserted into a bladder 2 in a patient. The balloon 13 of the catheter 1 is inflated to retain the bladder-dwelling distal end section A of the catheter body 11. At this time, the diagnosis section B of the catheter body 11 is disposed on an affected area near an inlet port of the bladder 2. The lead wires 15 extending from the proximal end of the catheter 1 are connected to an input terminal 41 of the current perception threshold inspection apparatus 4. A power source 43 and a display 42 are connected to the apparatus 4.

A feeble alternating current with a given frequency (5 to 2000 Hz) is supplied from the current perception threshold inspection apparatus 4 to the lead wires 15 of the catheter 1. The current flows through the electrodes 14 into the affected area in the bladder 2. The display 42 indicates a current level at the time when the patient feels the current flow. FIG. 7 shows an example of an indicating result of the current flow feeling-level. FIG. 7(A) shows a normal value while FIG. 7(B) shows an abnormal value due to the interstitial cystitis. In FIG. 7, the horizontal axis represents the frequency of the current applied to the electrodes. On the other hand, the vertical axis represents the number of individuals who had a sense of being oppressed, a sense of being uncomfortable or a sense of having a pain from the applied current. Those patients suffering from the interstitial cystitis are known that they are typically perceptible to a current at a lower frequency as compared to those in good health. As seen in FIG. 7(A) representing data for those individuals in good health, many of them have the sense of being oppressed at the frequency in a range of 17 Hz to 21 Hz. On the other hand, the patients suffering from the interstitial cystitis have the sense of being oppressed at the frequency in a range of 5 Hz to 9 Hz. This demonstrates that using the catheter of the present invention for making a diagnosis on a disease of a certain patient to verify the frequency level at which the patient has the sense of being oppressed makes it possible to determine if the patient actually being suffered from the interstitial cystitis or not at a high probability.

### Embodiment of the Present Invention

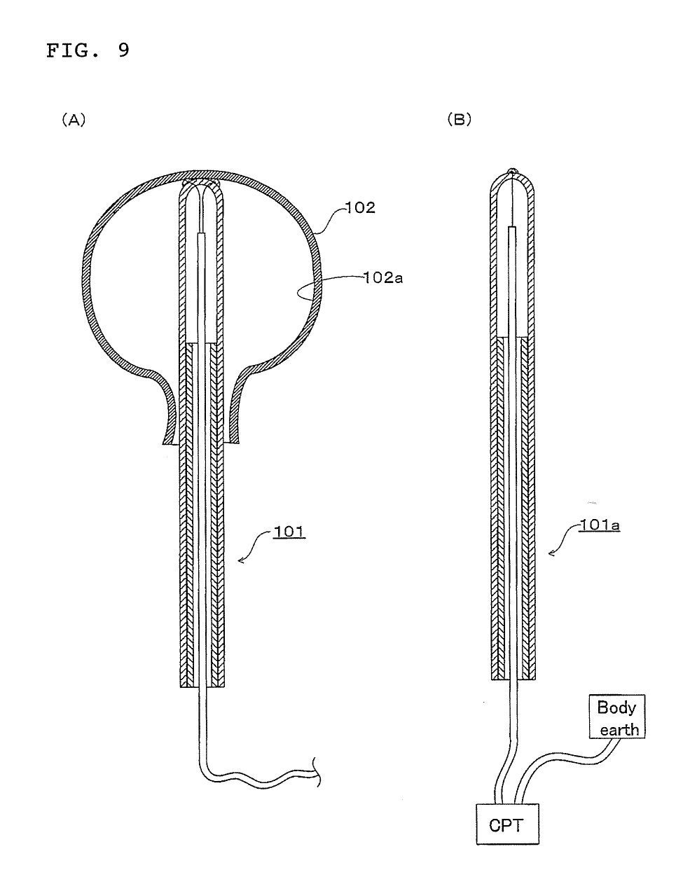

Referring now to FIGS. 8 to 13, a diagnosis catheter for an interstitial cystitis according to an embodiment of the present invention will be described. It is to be noted that those elements provided with no specific description are similar to ones in the diagnosis catheter 1 for an interstitial cystitis as previously described by way of the reference example, and so any duplicated explanation should be herein omitted.

A diagnosis catheter 101 for an interstitial cystitis comprises primarily a catheter body 111, a core member 112, a pair of electrodes 114 and lead wires 115, as shown in FIGS. 8(A) and 8(B).

The catheter body 111 is made of a soft flexible material (for example, silicon rubber) and has a distal end section A to be inserted into the bladder and a body section B disposed adjacent to the distal end section A and defined in a proximal end side. The core member 112 is made of a hard flexible material (for example, polytetrafluoroethylene resin) and adapted to be inserted into the catheter body 111 up to the vicinity of a lower end of the distal end section A.

The pair of electrodes 114 are provided on a distal end of the distal end section A of the catheter body 111. The leads 115 are inserted into the core member so that first ends of the lead wires are connected to the electrodes 114 and second ends of the lead wires are drawn out of a proximal end section of the catheter body 111 to be connected to the current perception threshold inspection apparatus 4 (see FIG. 6).

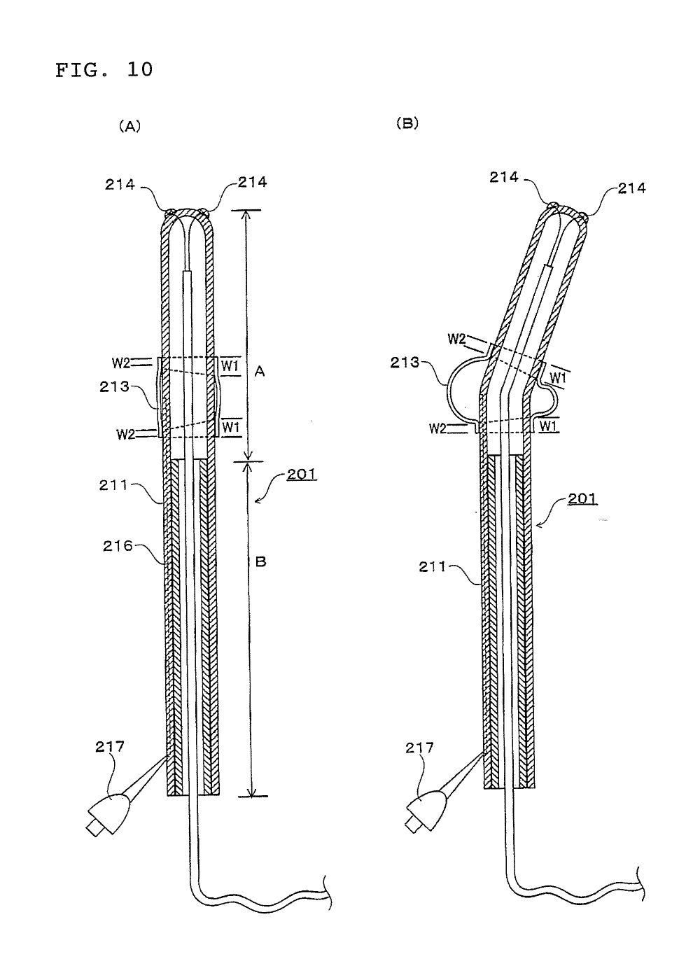

FIG. 9(A) shows the catheter 101 as disclosed in FIG. 8 having been inserted in the bladder 102. As seen from the FIG. 9(A), the catheter 101 has been inserted into the bladder until its distal end comes into contact with an interior wall 102a of the bladder 102. As it is placed in position, a feeble alternating current at a predetermined frequency (5-2000 Hz) is applied from the current perception threshold inspection apparatus 4 through the lead wires 115 of the catheter 101. The current flows from the electrodes 114 of the catheter 101 to the interior wall 102a of the bladder 2. The current level at which the patient has become aware of the current conduction is indicated on the display 42 (see FIG. 6). Accordingly, the diagnosis of the interstitial cystitis can be thereby made in a similar manner to that described in the reference example.

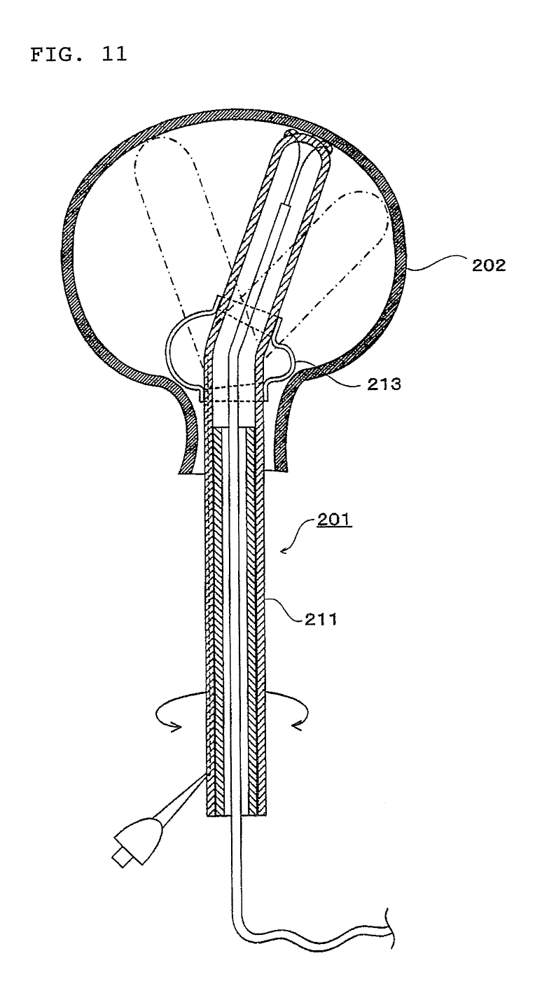

FIG. 9(B) shows a catheter 110a representing a modification of the catheter of FIG. 9(A). The catheter 101a includes one electrode provided on the distal end of the catheter body and another electrode provided as a body earth connected to the CPT. In an actual practice, the catheter 101a is inserted into the bladder of the patient with a disease to be diagnosed, while at the same time the body earth is brought into contact with some region on the patient body. This can create a closed circuit between the electrodes, so that the diagnostic procedure can be carried out as in the case of the catheter 101 of FIG. 9(A). It is needless to say that the above modification is similarly applicable to the type of catheter which will be described in connection with FIG. 10 and others following to FIG. 10.

### Second Embodiment

Referring now to FIGS. 10 to 13, a diagnosis catheter 201 for an interstitial cystitis according to a second embodiment of the resent invention will be described. The catheter 201 according to this embodiment is different from that of the first embodiment in that the catheter 201 has a specific balloon 213 provided on an exterior surface of a catheter body 211. Specifically, a cylindrical balloon 213 is fitted over the catheter body 211 at its lower end of a distal end section A so as to cover the exterior surface of the catheter body 211. The balloon 213 adheres in its upper and lower ends to the exterior surface of the catheter body 211 by using an adhesive. In this connection, a width of the adhesion varies among locations along the circumferential direction. Specifically, as shown in FIG. 10(A), the adhesion in one side (the right-hand side in FIG. 10(A)) with respect to the diametrical direction of the catheter body 211 is provided with a width of adhesion W1. The adhesion in the other side (the left-hand side in FIG. 10(A)) with respect to the diametrical direction of the catheter body 211 is provided with a width of adhesion W2. That is to say, the width of adhesion varies continuously from W1 to W2 circumferentially along the exterior surface.

In addition, an injection section 217 is provided in a proximal side of the catheter body 211. The injection section 217 serves to inflate the above-described balloon 213. Further, a fluid supply passage 216 is formed inside the cylindrical wall constructing the catheter body 211 to provide a fluid communication between the injection section 217 and an inside space of the balloon 213. This allows a fluid (for example, air, water and the like) to be supplied through the fluid supply passage 216 into the balloon 213 for inflating the balloon 213.

In the catheter 201 having a configuration as described above, once the fluid is supplied from the injection section 217, the balloon 213 starts to expand. At that time, since the width of adhesion of the balloon 213 is not uniform, a degree of expansion varies depending on the location as shown in FIG. 10(B). Referring to the illustrated embodiment, since the right-hand side of the balloon 213 has the relatively large width of adhesion, meaning that the balloon 213 is restricted by a larger area in the right-hand side, consequently the right-hand side of the balloon 213 permits a smaller degree of expansion than the left-hand side of the balloon 213. In this regard, since the catheter body 211 is made of a soft flexible material, the catheter body 211 is allowed to deform under any external force applied thereto. In this manner, the balloon 213 tends to expand ununiformly with respect to the catheter body 211 of the soft flexible material and thus the catheter body 211 is subject to an ununiform external force. Consequently, such ununiform external force causes the catheter body 211 to bend, as shown in FIG. 10(B).

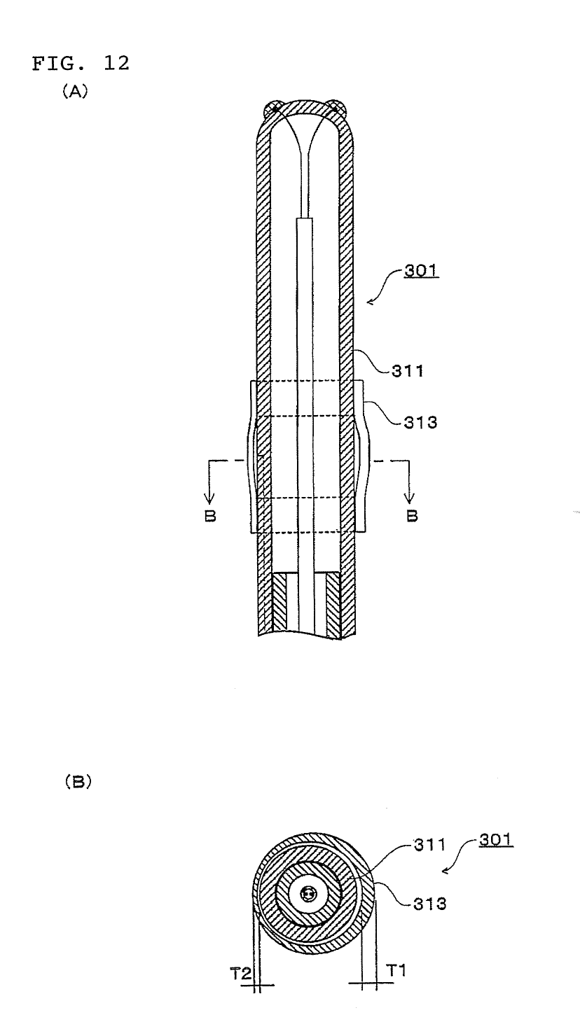

FIG. 11 shows a diagnostic procedure being carried out on the interior wall of the bladder 202 by using the catheter 201 as shown in FIG. 10. Since the catheter body 211 is bent as illustrated, it is possible to apply the diagnostic procedure to other sites than the topmost region of the interior wall of the bladder 202. In addition, the diagnostic procedure can be applied to much extended area by rotating the catheter body 211 around the longitudinal axis as a center of rotation. It is to be especially noted that by changing a volume of the fluid to be injected into the balloon 213 (for example, 1 cc to 4 cc), the external force applied to the catheter body 211, and thus, the angle of the bending of the catheter body 211 may be changed (for example, 30 degrees to 50 degrees). This allows a desired diagnostic procedure to be carried out in the sites of the bladder interior wall. As noted above, the catheter is not bent when no fluid is injected into the balloon. Accordingly, it facilitates the insertion of the catheter into the bladder.

### Variation 1

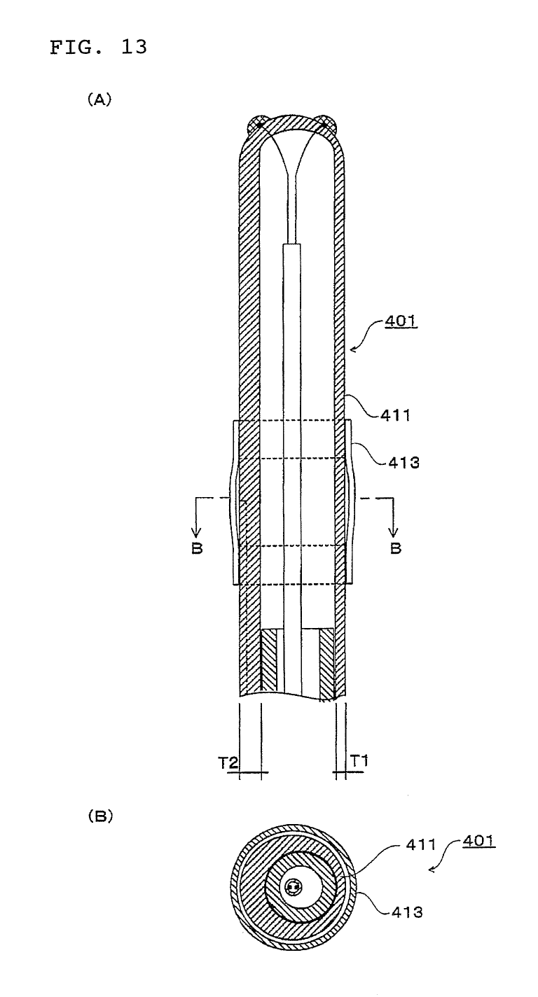

FIG. 12 shows a variation of a diagnosis catheter for an interstitial cystitis according to the second embodiment. In the illustrated variation, a balloon 313 has a different structure. Specifically, the balloon 313 has a varying thickness dependent on the location. The balloon 313 has a greater thickness T1 in the right-hand side than a thickness T2 in the left-hand side, as illustrated. Owing to this, when the fluid is injected into the balloon 313, the balloon 313 tends to expand larger in the left-hand side than in the right-hand side. Consequently, the external force applied to the catheter body 311 is also ununiform, and thus the catheter body 311 is biased to bend. In this connection, preferably, the angle of the bending of the catheter body 311 can be set correspondingly to the volume of the fluid to be injected. Specifically, it is advantageous that the catheter body 311 can bend at the bending angle in a range of about 30 degrees to 50 degrees as the volume of the fluid injected into the balloon increases in a range of 1 cc to 4 cc.

### Variation 2

FIG. 13 also shows another variation of a diagnosis catheter for an interstitial cystitis according to the second embodiment. In the illustrated variation, a catheter body 411 has a different structure. Specifically, the catheter body 411 has a varying exterior wall thickness dependent on the location. The catheter body 411 has a reduced thickness T1 in the right-hand side as compared to a thickness T2 in the left-hand side, as illustrated in FIG. 13. Owing to this, when the fluid is injected into a balloon 413, catheter body 411 tends to become deformed more significantly in the right-hand side than the left-hand side. Consequently, even if the external force applied from the balloon 413 is acting uniformly on the catheter body 413, the catheter 411 still can bend.

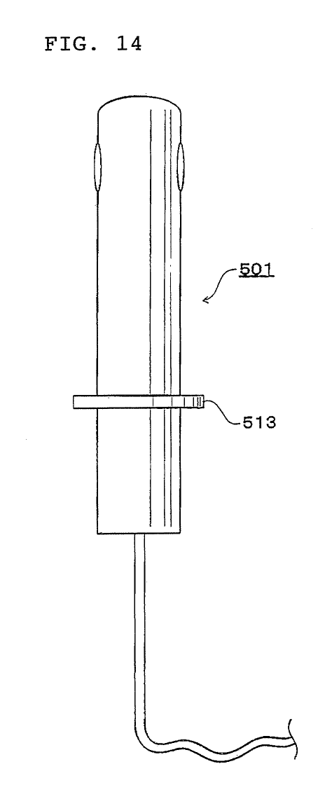

FIG. 14 shows a catheter with an electrode provided on a lateral surface of a catheter body. This catheter 501 is intended to support an application in the vagina but not for the diagnostic procedure in the bladder. To this end, the catheter 501 has a larger body size and has an annular flange 513 for controlling a insertion depth of the catheter body. The diagnostic procedure, if carried out in the bladder by using the catheter prepared specifically for the diagnostic procedure in the bladder in parallel with the diagnostic procedure carried out in the vagina by using the catheter prepared specifically for the application in the vagina, can make an improved diagnosis of interstitial cystitis with high precision.

### Possibility of Utilization in an Industrial Field

According to the present invention, it is possible to simply and precisely diagnose an interstitial cystitis by using a catheter having a simple and inexpensive structure. The catheter does not cause a pain in a subject during inspection. The catheter of the present invention will be useful for a diagnosis of an irritable bowel syndrome.

1. A diagnosis catheter for an interstitial cystitis wherein the catheter is connected to a current: perception threshold inspection apparatus to diagnose the interstitial cystitis, comprising:
a cylindrical catheter body made of a soft flexible material and including a distal end section to be left in the bladder and a body section disposed adjacent to said distal end section and defined in a proximal end side;
at least one electrode provided on a distal end of said catheter body; and
a lead wire inserted into said catheter body so that first end of said lead wire is connected to said electrode and second end of said lead wire is drawn out of a proximal end of said catheter body to be connected to said current perception threshold inspection apparatus.

2. A diagnosis catheter for an interstitial cystitis according to claim 1, characterized in further comprising: a balloon mounted on an outer periphery of said catheter body near a boundary between said distal end section and said body section; a fluid supply passage formed inside said catheter body in communication with said balloon; and an injection section capable of injecting a predetermined fluid through said fluid supply passage.

3. A diagnosis catheter for an interstitial cystitis according to claim 2, characterized in that both end sections of said balloon adhere to an exterior surface of said catheter body, and a width of adhesion varies circumferentially along said exterior surface of said catheter body.

4. A diagnosis catheter for an interstitial cystitis according to claim 2, characterized in that said balloon has a varied thickness circumferentially along said exterior surface of said catheter body.

5. A diagnosis catheter for an interstitial cystitis according to claim 2, characterized in that said catheter body has a varied exterior wall thickness circumferentially along said exterior surface thereof.

6. A diagnosis catheter for an interstitial cystitis according to claim 3, characterized in that said catheter body has a varied exterior wall thickness circumferentially along said exterior surface thereof.

7. A diagnosis catheter for an interstitial cystitis according to claim 4, characterized in that said catheter body has a varied exterior wall thickness circumferentially along said exterior surface thereof.
